(19) United States
(12) Patent Application Publication
(10) Pub. No.: US 2021/0371845 A1
(43) Pub. Date: Dec. 2, 2021

(54) Synthesis Of Enzyme-Polymer Conjugates, Having Enhanced Activity & Stability

(71) Applicant: Nestor Maceda-Johnson, SW Ranches, FL (US)

(72) Inventor: Nestor Maceda-Johnson, SW Ranches, FL (US)

(21) Appl. No.: 16/886,254

(22) Filed: May 28, 2020

(51) Int. Cl. C12N 11/082

(52) U.S. Cl. CPC C12N 11/082

(57) ABSTRACT

A process for site-specific immobilization of an enzyme on a polymer involving pairing of an enzyme and polymer to optimize cross-linking between the enzyme and the polymer while avoiding conformational or biochemical inhibition of enzyme activity. The process involves site specific interaction in a multiple phase synthesis within a buffered reaction medium and in an inert atmosphere. The reaction kinetics of the conjugation are modulated by chilling the reaction medium containing the enzyme and polymer to about the maximum solvent density of the reaction medium. The cross-linking of the enzyme and a polymer pair performed produces an enzymatically active, stable conjugate.

### BACKGROUND OF THE INVENTION

### 1. Field of the Invention

This invention relates to methods for preparation, (also “synthesis”), and use of enzyme-polymer conjugates, and compositions incorporating such conjugates. More specifically, the synthesis and methods of use of this invention relates to preparation of enzyme-polymer conjugates, having both enhanced activity and stability, resulting from optimizing enzyme to polymer binding/cross-linking.

### 2. Description of the Prior Art

The interaction of polymers and enzymes to form enzymatically active conjugates is well-known, and can typically result in the immobilization of the enzyme upon the polymer. These interactions can be random, or site specific. Numerous enzymes of biotechnological importance have been immobilized on various supports (inorganic, organic, composite and nanomaterials) via random multipoint attachment. However, immobilization via random chemical immobilization results in a heterogeneous protein population, where more than one side chain, (amino, carboxyl, thiol etc.), present in proteins (enzyme), is linked with the support, resulting in potential reduction in enzyme activity due to restriction of substrate access to the active site of the enzyme.

In contrast to the random chemical modification/immobilization of an enzyme, site-directed enzyme immobilization (also “SDEI”) is preferred because a polymer is linked to a single specific amino acid (generally N- or C-termini) remote from the active-site of the enzyme. In site specific immobilization, maximal enzyme activity is retained due to the free access of a substrate to the active-site on the immobilized enzyme.

These strategies are mainly chemically driven/directed and may additionally require genetic and enzymatic methods to generate functional groups (that are absent in protein/enzyme) for interaction between the support and the enzyme. Selection of the SDEI method depends on many factors, such as the type of enzyme (less stable psychrophilic, or more stable thermophilic homologue, for example), pH stability of enzyme, the availability of N- or C-termini to the reagent, non-interference of the enzyme terminus with the enzyme activity, type of catalytic amino acid residue, and the availability, price and the ease of preparation of reagents.

Notwithstanding the disclosure of various published procedures for synthesis of enzyme/polymer conjugates, such synthesis has been and remains fraught with uncertainty because of the sensitivity of the enzyme to the variables arising in conjugate synthesis, the effect of such variables upon the reaction kinetics in such synthesis (affinity of the enzyme to binding sites upon the polymer, for example), and the complex internal dynamic motions of functional groups within the enzyme.

Accordingly, there continues to exist a need for a more reproducible process for the synthesis of enzyme/polymer conjugates wherein such conjugates have and retain enhanced activity and, further, the enzyme of the activity of the conjugate is more stable and sustainable under applications having diverse environmental conditions.

### OBJECTIVES OF THIS INVENTION

It is the object of this invention to remedy the above, as well as related deficiencies, in the prior art.

More specifically, it is the principle object of this invention to provide a proprietary method for the synthesis of stable and enzymatically active polymer conjugates, for industrial, diagnostic and medical applications.

It is an object of this invention to provide a proprietary method for the synthesis of stable and enzymatically active polymer conjugates essentially free of steric hindrance of the enzyme.

It is still yet another object of this invention to provide a proprietary method for the synthesis of stable and enzymatically active polymer conjugates by affinity, site specific interaction of the polymers to enzymes.

It is still yet another object of this invention to provide a proprietary method for the synthesis of stable and enzymatically active bio-conjugates, by judicious pairing of enzyme and polymer, by interactive sites, to attain site specific interaction of the polymers to enzymes.

It is still yet another object of this invention to provide a proprietary method for the synthesis of stable and enzymatically active bio-conjugates, by judicious pairing of molecular weight to minimize polymer encapsulation of the enzyme, and thereby effectively inhibit steric interference with the of the active site of the enzyme.

Additional objects of this invention include providing a proprietary method for the synthesis of stable and enzymatically active polymer conjugates with increased stability (under the harsh conditions used in industrial process uses) for medicinal uses, food processing, filtration and separation processes, energy sequestration and storage, cosmetics and skin care.

### SUMMARY OF THE INVENTION

The above and related objects of this invention are accomplished with the following synthesis (also “methods”), as herein described, for the preparation of unique enzyme-polymer conjugates (also “bio-conjugates” or “bioconjugates”), specifically, conjugates having both enhanced enzyme activity and enhanced enzyme stability. This process contemplates the site-specific immobilization of enzyme on polymers, involving judicious selection (pairing) of an enzyme and a polymer (also “cross-linker”) combination, to optimized interaction between the enzyme and the polymer under the conditions of the synthesis of this invention while avoiding physical (conformational) and/or biochemical inhibition of enzyme activity.

In the preferred embodiments of this invention, the interaction of enzyme and polymer is modulated, to effectively prevent oversaturation and/or encapsulation of the enzyme molecule within the polymer. This modulation is accomplished by judicious pairing of enzyme and polymer of similar molecular weights, by conducting the interaction of these reactants with one another in multiple phases, and retarding the reaction kinetics by reducing the temperature of the reaction medium, to affect its maximum solvent density.

More specifically, in the initial phase of the method, each of the polymer and enzyme are reacted with each other in an appropriate (pH buffered) solvent (for preservation of enzyme activity) and in an inert atmosphere under cross-linking conditions. The relative concentration of polymer to enzyme in the reaction medium is in the range of about 1:1, based upon the number of available interactive cross-linking sites. Each of the polymer and enzyme is thus preferably present in stoichiometric concentrations. The reaction medium comprises a buffer which is selected based upon the enzyme compatibility. Some enzymes require an alkaline buffered environment, while others require an acidic buffered environment; such environments being generally determined from the organism of origin of the enzyme. In this initial phase of this method, an enzyme of a reactant pair is initially dissolved in a suitable, chilled buffer within a suitable reaction vessel (e.g. centrifuge tube). A polymer of a reactant pair is thereafter combined with the cold enzyme solution while stirring the reaction medium. The rate of polymer addition to the enzyme solution is metered so as to avoid clumping of the polymer. Upon combining the enzyme and polymer pair in the reaction medium, the contents of the reaction vessel are continued to be chilled and subjected to essentially continuous mild/gentile agitation in a refrigerated centrifuge. The combination of the gentile agitation and the chilling of the reaction medium (preferably to its maximum solvent density) modulates the rate of interaction (the reaction kinetics) of the enzyme and the polymer. The interaction of the polymer and enzyme pair reaches equilibrium (K₁) within a relatively brief interval (60 minutes, for example). Unreacted polymer thereby remains in solution and the resultant conjugate formed settles/separates from the reaction medium by settling to the bottom of the reaction vessel. As above noted, the chilling of the reaction medium increases its solvent density. The resultant increase in solvent density (also “solvent effect”) reduces the rate (also “reaction kinetics”) of interaction of the enzyme and polymer and, under the reaction conditions of this invention, effectively directs or limits such interaction of only one polymer molecule to one enzyme molecule. The formation of the desired conjugate, as above described, effectively prevents steric inhibition of the resultant enzyme-polymer (e.g. oversaturation of the enzyme with polymer and/or clumping), under the reaction conditions of this synthesis.

The essentially continuous, mild agitation of the reaction medium not only effectively mixes the reactants but also contributes to the modulated interaction of the polymer and enzyme to form a stable, enzymatically active polymer conjugate. The duration of the reaction interval is empirically determined by the reaction/cross-linking equilibrium, and such equilibrium is generally attained within from about 20-60 minutes.

In the second phase of the method of this invention a second enzyme solution is separately prepared by dissolving additional enzyme of the reactant pair in an identical reaction medium (chilled buffer). This second enzyme solution comprises an amount of enzyme equivalent to the amount of enzyme used in the initial phase of the interaction of the enzyme and polymer pair. The volume of this second enzyme solution is about two (2) times the volume of the reaction medium containing the enzyme/polymer bio-conjugate and unreacted polymer. Each of reaction medium containing the bio-conjugate and unreacted polymer and the second enzyme solution are combined, by the modulated addition of the second enzyme solution to the reaction vessel containing the enzyme/polymer bio-conjugate and unreacted polymer. The contents of the combined solutions are once again further reacted, under crosslinking conditions, (as above described for the initial preparation of the bio-conjugate). After mild agitation for about 20 to 60 minutes, the cross-linking of the additional enzyme and the free polymer reaches equilibrium. The contents of the reaction vessel are thereafter stored for use in one of more applications.

The addition of the enzyme to the polymer in two (2) distinct phases as above described yields a bio-conjugate having site specific immobilization of the enzyme on the polymer while having enhanced enzyme stability and activity when compared to a random or an unmodulated interaction of the enzyme and polymer.

### DETAILED DESCRIPTION OF INVENTION

As understood within the context of this invention, the following terms and phrases are intended to have the following art recognized meaning, unless otherwise indicated.

### Glossary of Terms

The term “enzyme,” as used herein, refers to globular proteins, whether acting alone or in larger complexes. The enzymes suitable for use in the synthesis and method of this invention are preferably water soluble, have a neutral charge, or neutral pH, and one or more of the following functional groups: —OH, —COOH, —SH, —SO, —CO, —H, and/or —NHx. Representative enzyme categories suitable for conjugation and stabilization with the method of this invention, and exhibiting the following functionality, include: proteinases, kinases, proteases, laccases, peroxidases, polymerases/transferases, and oxidoreductases. In practice, the molecular weight of the enzyme used in the method of this invention is preferably anywhere from 5 to 10 times the molecular weight of the polymer selected for site specific interaction with the enzyme. Such range is selected based upon maintaining the solubility in the reaction medium (buffer). Accordingly, the preferred relative molecular weight of the polymer can vary, depending upon its relative solubility and, therefore, may exceed this preferred range.

The sequence of the amino acids of the enzyme specifies the protein structure which in turn determines the catalytic activity of the enzyme. It is understood that enzymes are not rigid, static structures; instead they have complex internal dynamic motions—that is, movements of parts of the enzyme's structure such as individual amino acid residues, groups of residues forming a protein loop or unit of secondary structure, or even an entire protein domain. These motions give rise to a conformational ensemble of slightly different structures that interconvert with one another at equilibrium. Different states within this ensemble may be associated with different aspects of an enzyme's function.

Although an enzyme's structure generally determines its function, a novel enzymatic activity cannot yet be predicted from structure alone because enzymes denature when heated or exposed to chemical denaturants, which disruption to the structure typically causes a loss of activity. Enzyme denaturation is normally linked to temperatures above a species' normal level, as well as lower or higher pH ranges to those of the species' normal pH.

The term “polymer,” as used herein, refers to a soluble macromolecule having repeating organic and/or organo-metallic structural units and which is soluble in a buffered reaction medium, which medium is also capable of dissolving an enzyme without destabilization of the enzyme. The polymers suitable as an enzyme immobilization substrate/platform have site-specific affinity for interaction (also “pairing”) with an enzyme (also “paired polymer”). In practice, polymers suitable for use in the methods of this invention are liquids or low-melting solids, depending on their molecular weights. Accordingly, the polymers suitable for use in the methods of this invention (to pair with an enzyme) have a molecular weight of about 25,000 g/mol or less.

The pairing of the enzyme and polymer in the synthesis of an enzymatically stable conjugate pursuant to this invention is generally application specific (and determined by the use of the conjugate). More specifically, as a general guideline, the polymer pair should preferable have two (2) binding sites for each binding site on said enzyme. Moreover, the polymer pair needs to have a molecular weight of 25,000 g/mol or less, to ensure solubility in the reaction medium (e.g. the buffer) containing the enzyme and to remain dissolved in the reaction medium under the process conditions of this synthesis (i.e. at depressed reaction temperatures). In the preferred synthesis of this invention, the polymer pair of the enzyme-polymer conjugate should also be compatible with relatively harsh industrial processes and resistant to environment stresses.

Representative immobilization polymers (in the form of a liquid or low-melting solid) that can be paired with enzymes in the synthesis of the conjugates pursuant to this invention include a plurality of homopolymers and co-polymers. Suitable pairing of a homopolymer and an enzyme can include materials comprising aldoximes, ketoximes, muco-adhesion monomers, polyethylene glycol, bis-pyridinium oximes, N,N-dimethylacrylamide, N-isopropylacrylamide, (meth)acrylate, N,N-dimethylaminoethyl methacrylate, carboxyl acrylamide, 2-hydroxylethylmethacrylate, N-(2-hydroxypropyl)meth-acrylamide, quaternary ammonium monomers, sulfobetain methacrylate, oligo(ethylene glycol)methyl ether methacrylate, 2-PAM monomers, 4-PAM monomers, Clickable azide monomers, and combinations thereof.

Suitable pairing of a co-polymer and an enzyme can include materials comprising at least two different monomers wherein at least one monomer comprises aldoximes, ketoximes, muco-adhesion monomers, polyethylene glycol, bis-pyridinium oximes, N,N-dimethylacrylamide, N-isopropylacrylamide methacrylate, N,N-dimethylaminoethyl methacrylate, carboxyl acrylamide, 2-hydroxylethylmethacrylate, N-(2-hydroxypropyl) methacrylamide, quaternary ammonium monomers, sulfobetain methacrylate, oligo(ethylene glycol)methyl ether methacrylate, 2-PAM monomers, 4-PAM monomers, azide monomers, and combinations thereof.

The phrase “Polyethylene glycol” (“PEG”), as used herein, refers to polyether liquids or low-melting solids having the following molecular structure, H—(O—CH₂—CH₂)ₙ—OH. Polyethylene glycol is also known as polyethylene oxide (PEO) or polyoxyethylene (POE), depending on its molecular weight. PEG and PEO refer to an oligomer or polymer of ethylene oxide, chemically synonymous but historically the term PEG is preferred in the biomedical field whereas the term PEO appears more prevalent in polymer chemistry. Because different applications require different polymer chain lengths, PEG typically is used to refer to oligomers and polymers with a molecular mass below 20,000 g/mol; PEO to polymers with a molecular mass above 20,000 g/mol; and POE to a polymer of any molecular mass. PEGs are prepared by polymerization of ethylene oxide and are commercially available over a wide range of molecular weights, for example from 300 g/mol to 10,000,000 g/mol.

PEG and PEO exist as liquids or low-melting solids depending on their molecular weights. While PEG and PEO differ by molecular weight and have different physical properties (e.g. viscosity) due to their differing chain length effects, their chemical properties are nonetheless nearly identical.

The phrase “Coordination Polymer” (“CP”), as used herein, refers are liquids or low-melting solids having an inorganic or organometallic polymer structure containing metal cation centers linked by ligands. Within the context of this invention, a coordination polymer is a coordination compound with repeating coordination entities extending in 1, 2, or 3 dimensions. The term also describes a polymer where repeat units are coordination complexes. Coordination polymers contain the subclass coordination networks that are coordination compounds extending, through repeating coordination entities, in 1 dimension, but with cross-links between two or more individual chains, loops, or spiro-links, or a coordination compound extending through repeating coordination entities in 2 or 3 dimensions subclass of these are the metal-organic frameworks, or MOFs, that are coordination networks with organic ligands containing potential voids. In one of the preferred embodiments, an enzyme and coordination polymer pair (PEGMA) can be represented as follows:
(-[-enzyme-PEGMA-tmMOF-PEGMA-]ₙ-)

wherein PEGMA is polyethylene glycol methacrylate and tm is transition metal¹ (e.g. silver) within a metal organic framework of the coordination polymer,

The phase “Metal-Organic Frameworks,” or “MOF,” as used herein, refers subclass coordination networks that are coordination compounds extending, through repeating coordination entities in one dimension, but with cross-links between two or more individual chains, loops, or spiro-links, or a coordination compound extending through repeating coordination entities in two or three dimensions. Coordination polymers can be classified in a number of different ways according to their structure and composition. One important classification is referred to as dimensionality. A structure can be determined to be one-, two- or three-dimensional, depending on the number of directions in space the array extends in. A one-dimensional structure extends in a straight line (along the x axis); a two-dimensional structure extends in a plane (two directions, x and y axes); and a three-dimensional structure extends in all three directions (x, y, and z axes).

The term “Conjugate” or “Bio-Conjugate” or “Bioconjugate,” as used herein, refers to the site-specific association of an enzyme with a polymer having multiple functional sites and having an affinity for interaction to a single specific amino acid (generally N- or C-termini) on the enzyme remote from the active-site of the enzyme. The Conjugates of this invention, having enhanced stability, can be used under demanding environments (hard conditions); specifically in industrial process uses, medicinal uses, food processing, filtration and separation processes, energy sequestration and storage, cosmetics and skin care, and among any additional commercial uses for enzymes, not heretofore practical. In addition, the enzyme/polymer conjugates of this invention are also potentially suitable for use in anti-microbial/anti-biofouling coatings and paints; textile processing; and, in combination with metal-organic frameworks, or MOFs, for energy sequestration-battery formulations and oil/gas extraction and clean up.

### Synthesis of Conjugate” or “Bio-Conjugate” or “Bioconjugate

The Conjugate or Bio-Conjugate or Bioconjugate (collectively “Conjugate” or “Conjugates”) of this invention can be prepared by site-specific interaction (immobilization) of an enzyme with a paired polymer. The polymers used in this synthesis can be easily synthesized and/or readily available commercially. These starting materials (polymers and enzymes) are conjugated with each other by conventional means and methods in accordance with the sequence of steps and under the process conditions, as set forth herein.

The enzymes and polymers used in this synthesis can be combined, in the appropriate proportions, in a suitable reaction medium (e.g. buffered aqueous solution) and under cross-linking conditions. As above noted, the synthesis of this invention is suitable for the synthesis of conjugate having enhanced enzyme activity and stability. This synthesis is accomplished by site specific interaction of paired enzymes and polymers, under cross-linking, in at least two (2) discrete stages.

The selection of suitable reaction medium (e.g. buffered aqueous solution) is chosen for its compatibility with the enzyme, to insure the stability of the enzyme, specifically preservation of its macromolecular structure and therefore its enzyme activity. In the preferred embodiments of this invention, the buffer consists of a mixture of a weak acid and its conjugate base, or vice versa, to maintain the pH of the reaction medium at a nearly constant value (in the range of about 7 to about 8). The preferred reaction medium, suitable for use in this invention includes: TRIS, sodium acetate, sodium citrate, disodium phosphate, potassium phosphate, SDH, DAN, Sodium bicarbonate, sodium oxalate, and ammonium nitrate.

In the initial stage of the preferred embodiment of this invention, an enzyme is dissolved in a reaction medium having a pH optimized for the enzyme (e.g. TRIS buffer adjusted to optimal pH, based upon biological source of enzyme). The relative concentration thereof in the reaction medium is less than the maximum quantity of enzyme and polymer that can dissolve in a given quantity of reaction medium within the range of temperature conditions prevailing under the conditions of the synthesis of the conjugate.

The polymer pair (also “paired polymer”) is added to the reaction medium containing the dissolved enzyme under immobilizing/cross-linking conditions. The rate of addition of the polymer pair to the enzyme solution is metered/modulated to avoid encapsulation of the enzyme and clumping of the paired polymer in the reaction medium. In the preferred embodiments of this invention, the reaction vessel is a centrifuge tube which is placed in a centrifuge wherein the centrifuge is temperature controlled. The mole ratio, or relative amount, of each of the polymer pair to enzyme is based upon the number of potential binding sites on the polymer pair relative to the enzyme and the extent to which the relative amount of each of the polymer pair to enzyme steric may inhibit enzyme access to the binding sites on the cross-linker. In this initial phase of the synthesis, the mole ratio of polymer pair to enzyme is 1:1, based upon the number of potential binding sites on the polymer pair relative to the paired enzyme.

In one of the preferred embodiments of the synthesis, the site-specific immobilization of enzyme with a paired polymer is performed in multiple phases. More specifically, the modulation of the interaction of the enzyme and a paired polymer, as above described, is initially allowed to proceed to an equilibrium. At that juncture, additional paired polymer, in a second buffer solution, is added to the centrifuge tube containing the enzyme-polymer conjugate. The rate and manner of addition of the polymer in a second buffer solution is carefully metered to avoid physical encapsulation (e.g. clumping) and/or biochemical inhibition of enzyme activity. In this second phase of the conjugate synthesis, the second solution containing a polymer pair (e.g. PEG) is prepared by first dissolving the polymer in a second buffer solution (in the desired proportion as the initial reaction medium) and then also added to the centrifuge tube containing the conjugate. The resultant solution/dispersion is further agitated by centrifugation at a reduced temperature (˜0 to 4 degrees centigrade, for example), and at a relatively slow speed (˜500 to 1000 RPM, for example) to modulate the rate of binding of the enzyme to the polymer pair to effect the controlled formation of the desired conjugate without precipitation of the reactants. The multiple stage site specific interaction of the polymer pair with the enzyme, at reduced temperature, progressively results in the further site specific binding between the enzyme and the polymer pair.

At the reduced temperature prevailing during such site specific immobilization between enzyme and polymer pair, the relatively density of the reaction medium effectively retards the rate (reaction kinetics) of the site specific binding (e.g. cross-linking) reaction. The combination of mild agitation and reduced temperature induces reaction kinetics which favors the site specific interaction of enzyme and polymer pair, while avoiding the encapsulation of the enzyme by the polymer pair and clumping of the polymer when added to the enzyme solution. The site specific interaction of enzyme and polymer pair and formation of the conjugate in the reaction vessel reaches equilibrium during this second phase of the synthesis, after about 60 minutes. The conjugate, prepared as above described, has enhanced activity and stability when compared to conjugate prepared via random chemical immobilization techniques. The synthesis of the desired conjugate is confirmed by spectrophotometry and such spectrofluorometric assay confirms the extent of the site specific binding between enzyme and the polymer pair.

Where additional site specific binding is desired, the second phase of the process can be repeated, so long as the enzyme activity of the desired conjugate is not adversely effected.

### EXAMPLES

The Examples set forth hereinafter describe a number of illustrative processes for the synthesis of the conjugate(s) in accordance with the combination of procedures and conditions of this invention. The equipment and materials used in these Examples are readily available or can be constructed or prepared from readily available parts and materials. Where not otherwise noted and described, parts and percentages are by weight unless otherwise indicated.

### Example I

### Textile Preparation

1) Preparation of polymer-silver solution
   a) Prepare 15 percent weight solution of 80 kDa polycaprolactone (PCL) in DMF solvent. PCL obtained from Sigma-Aldrich.
      i) Different polymers can be used but for this example, PCL was used as it allowed for a higher weight percentage in DMF solution compared to other options available at the time.
   b) Stir in spherical silver nano particles until reaching Silver concentration of 5% in final solution.
      i) Spherical nanoparticles were used instead of triangular to allow for better uniformity in electrospinning process. Silver was chosen for its anti-microbial properties as well as its anti-radiation abilities. In some variations, 5% silver nanoparticles can be exchanged for either 8% WSO₄ solution or 8% MoSO₄ solutions for better anti-radiation.
   c) Sonicate until solution is uniform and homogenous using either bath or probe sonicator at standard temperature and pressure.
2) Prepare stabilized protease enzymes using method described below:
   a) Equilibrate all liquid reagents to 4 degrees Celsius prior to reactions.
      i) Lowering the temperature of the reaction allows for a larger margin of error and slower reaction times when working with enzymes in the lab. Lowering all reagents to 4 degree Celsius allows for slower rise in temperature when mixing reagents.
   b) Weight out maleimide-peg-succinimidyl valerate, (MAL-PEG-SVA 20,000 MW), obtained from LaysanBio, under Nitrogen gas atmosphere and place in individual vials.
      i) This specific PEG was used in order to save time, but silica treated PEG can also be made in the laboratory through a series of steps. The weighing out of the PEG and placing in individual vials was done under nitrogen gas as a preventative measure to prevent any unwanted oxidation of the treated PEG. The 20,000 was chosen specifically to keep the MW of PEG and Enzyme as close to 1:1 ratio as was possible.
   c) Calculate the number of millimoles (mMole) of PEG available in each of the pre-dispensed vials by dividing the mg in the vial by 20,000.
      i) This calculation was done to calculate the moles of PEG reacting in the vials.
   d) Calculate the amount of mg of Protease enzyme using the ratio of 28000 mg/mMole.
      i) The mass of Protease enzyme was calculated in this step using a ratio of 1:1 from the mMoles of PEG obtained in the previous step.
   e) Let Protease adjust to room temperature before reacting.
      i) This allows the protease to become more acclimated to the environment, allowing for better retention of activity.
   f) Weigh out mg amount calculated previously, step 2.d.
   g) Dissolve the amount weighed out in step 2.f in 1.5 mL of 0.015M K-HPO₄ buffer at pH 7.0. (use a screw cap conical micro-centrifuge tube). Insert a magnetic stir bar in vial as well to stir the solution using a magnetic plate or stirrer.
      i) The buffer is chosen depending on the enzyme and its origin before purification. In this case the enzyme came from a slightly basic environment and thus the buffer used was neutral and pH was increased later by adding pH 8 buffers. A conical centrifuge vial is used for compatibility with centrifuge as well as providing a clearer view of reaction after centrifuging. Stir bar is used for gentle stirring and mixing and to avoid risk of contamination by mixing with other materials or stirring rod.
   h) Add powder MAL-PEG-SVA from step 2.b to the stirring solution in step 2.g and allow to fully dissolve.
      i) The PEG is added to the enzyme solution and not vice-versa to avoid clumping of PEG if too much is added to quick. It is added slowly to allow for the PEG to dissolve as it is added while stirring slowly.
   i) Insert centrifugal tube with cap closed and counter balance tube containing H₂O in centrifuge pre-set to 4 degrees Celsius. Spin slowly at 100 rpm for 1 hour. Used a Beckman-coulter Allegra 64r refrigerated centrifuge.
      i) Lowering the temperature to 4 degrees gives increased control over reaction as it lowers enzyme activity and bonding speeds of PEG to enzyme. Doing so while centrifuging forces part of the PEG towards the bottom of the vial, preventing over functionalizing the enzyme with the treated PEG.
   j) After 1 hour, stop centrifuge and remove the reaction tube. Add an equal volume of 0.20 M K-HPO₄ at pH 8.0, containing an additional amount of protease enzyme but not exceeding the amount weighed out in step 2.f. Activate magnetic stir bar again.
      i) Adding the pH 8 buffer brings the pH up from pH 7 closer to natural pH of the protease enzyme being used in this example. This solution containing more protease enzyme brings the ratio of enzyme to PEG up from 1:1 to close to 2:1. This is to keep the PEG under control from over functionalizing the enzyme and limiting its activity. The extra enzyme bonds with unreacted PEG that was pushed down while centrifuging. These control steps are critical for maintaining and increasing the activity of the functionalized enzyme.
   k) Replace cap on centrifugal tube, and rebalance with counter tube with water. Restart the centrifuge and spin at 100 rpm for 1 hour at 4 degrees Celsius.
      i) This step allows for the completion of the reaction between the PEG and enzyme. The centrifuge is kept at slow speed of 100 rpm and low temperature to lawyer the reaction where functionalized PEG-enzyme conjugates are towards the bottom, PEG in middle, and enzymes towards top based on weights. The low temperature slows the enzyme activity and molecular interactions enough for the reaction to occur in an organized manner.
   l) Stop centrifuge and remove cap from centrifugal tube and add a 2× volume of 0.20 M Glycine/0.20 M K-HPO₄ pH 8. Solution must be cold.
      i) The addition of cold glycine/K-HPO4 buffer is to again raise the pH closer to 8, should be around the 7.8-7.9 mark at this point to mimic the enzyme's environmental pH. This increase volume dilutes any unreacted PEG to negligible amounts and creates enough spaces between PEG and PEG-enzyme conjugate to avoid further reactions without going through separation methods. This is done as some separation methods can introduce impurities or cause further unwanted reactions with PEG. Solution is added cold to prevent sudden increase in temperature.
   m) Replace cap, rebalance centrifuge with counter tube, and restart the centrifuge at 100 rpm for 30 minutes.
      i) This again layers the solution to separated unreacted reagents from the product. At this point the PEG-enzyme conjugate is towards the bottom but still dissolved.
   n) After 30 minutes remove from centrifuge and analyze/record UV spectrum at 240-340 nm. Used a Shimadzu UV-Vis Spectrophotometer (UV-2600) with TCC-controller.
      i) This is to detect that the solution still has the enzyme, and to observe that there has been a shift in peak location after reaction with PEG. Standard enzyme UV peaks can be easily found for comparison, or one can be taken at the start.
   o) Enzyme activity can be checked after completion of steps 2.a-2.n by reacting the enzyme with its substrates or using an enzyme activity assay kit available by many manufacturers. For our purposes we used Promega ADP-Glo assay kits. Used a Synergy H2 microplate reader in luminescence mode for assays.
      i) This is done to prove the enzyme is still active and compare activity to unreacted enzyme standards.
3) Add in hexagonal-Boron Nitride into solution until reaching 15% h-BN solution concentration.
   a) h-BN is added in the solution to provide anti-radiation and anti-IR properties due to its ability to absorb excess energy via electron displacement. h-BN also provides some anti-microbial properties to complement those of the protease enzyme in the finished textile. The use of h-BN or other 2-D materials also provides a better structure with increased uniformity in electrospinning or deposition methods.
4) Mix final solution while sonicating or mixing lightly until homogenous. Leave in sonicator for approx. 2-3 hours.
   a) The final solution is right on limits of saturation so it may precipitate polymer conjugates if left standing for too long. Sonicating or mixing solution prevents precipitates from forming in the solution as these are not picked up by electrospinning pumps and left from final textile. If electrospinning is not done immediately, the solution can be stored and sonicated before electrospinning to get rid of any unwanted precipitates.
      Final solution for electrospinning is 15% PCL, 5% silver nano particles, 15% hexagonal-Boron Nitride, 2% modified protease enzyme.
         This solution is then taken to electrospinning if desired.

The electro spinner used in development of this invention was a Bioinicia Fluidnatek LE-100, others could prove successful as well, given the process is done at standard temperature and pressure.
Set flow rates to 500 uL/h.
Set a potential difference of 14 kV.
Set elevation to 200 mm.
Set rotational speed of collector drum at 50 mm/s
Set pump diameter to 12.45 mm.
Leave spinning until solution is run out.
Cut sample in straight line and peel from drum, to obtain a sheet. A double drum collecting method can be used to collect sample in a rolled form.

### Example II

### Conjugation of Enzyme-Polymer With MOF's and Uses

1. Polyethylene glycol (PEG) of roughly one-fifth the molecular weight of desired protease enzyme is treated with an acid to create polyethylene glycol methacrylate (PEGMA) monomer.
a. This was done for the sole purpose of creating a PEGMA monomer and save costs of purchasing pre-made PEGMA monomers. One can also start the synthesis with pre-purchased PEGMA.
b. Lower molecular weight is used compared to example 1 since this reaction will create a larger polymer at the end with a similar molecular weight to the protease enzyme used (protease).

2. The product of step 1, PEGMA is the reacted with a long-chain thiol containing carboxylic acid, in this case mercaptoundecanoic acid.
a. This step is done to attach a thiol (—SH) at one end of the polymer to serve as an attachment point for the protease enzyme.

3. The product of step two is then further reacted via radical mediated polymerization with either additional PEGMASH.
a. This step is done to create a polymer ligand to attach to the enzyme via the carboxylic group of the polymer and the thiol group of the protease enzyme.

4. The Protease enzyme is reacted as in Example I at this point with the exception of substituting PEGMA generated in this example for PEG used in Example I; And the substitution of a silver metalorganic framework (MOF) containing a thiol group¹ instead of silver nanoparticles used. Electrospinning is not done on this example.
a. This step is done to stabilize the enzyme as in Example I, but with a thiol functionalized polymer and a thiol functionalized silver MOF. This allows the joining of the treated protease enzyme-PEGMA-SH conjugate to the thiol silver MOF via thiol-ene reaction.
b. This generates the compound: -[-enzyme-PEGMA-AgMOF-PEGMA-]ₙ-

5. This solution is then covered in aluminum foil as a preventative measure until used in desired usage.

6. Analyze via Infrared spectrometry and NMR-1, compare to initial compound scans.

See also: https://doi.org/10.1039/C5NR01292A (which is incorporated by reference)
Usage of -[-enzyme-PEGMA-AgMOF-PEGMA-]ₙ- of this example:

The primary usage of this Example II is to create an enzyme-polymer-MOF conjugate that is highly anti-microbial, by combining the anti-microbial properties of protease enzymes with the anti-microbial properties of silver, in a single polymer. By combining the silver MOF into the polymer this also provide anti-radiation properties to this solution giving better stability.

This polymer can be left in liquid form for use as a protective and antimicrobial coatings, antiseptic solutions, or used/combined with polymer, or latex based paints, for additional anti-biofouling protection.

This Example II can be repeated using enzymes form the kinase, protease, proteinase, laccases, peroxidases, oxidoreductases, and polymerases/transferases classes.

### Example III

The Synthesis of Example 1 is repeated except for the substitution of enzyme Kinase for protease. Electrospinning does not need to be done for Kinase type enzyme.

### Example IV

The synthesis of Example 1 is repeated except for the substitution of enzyme type protease for enzyme type oxidase. pH range for this reaction is changed to 4-7. Electrospinning collection is changed from drum collection to plate-style collector for particles or beads instead of textile.

### Example V

The synthesis of Example 1 is repeated except for the substitution of polyacrylonitrile (PAN) for polycaprolactone (PCL).

### Example VI

The synthesis of Example 1 is repeated except for the substitution of Lipase type enzymes for protease enzymes. pH range for Lipase enzyme is kept between pH 7-10. Lipase enzyme does not need to undergo electrospinning.

### Example VII

The synthesis of Example 1 is repeated except for the substitution of nylon or any polyester type polymers for polycaprolactone.

### Example VIII

The synthesis of Example 1 is repeated except for the addition of graphene components to the solution at ranges of 5-25 percent weight in final solution.

### Example IX

The synthesis of Example 1 is repeated except for the substitution of polyethylene oxides (PEO) instead of polyethylene glycol (PEG) for molecular weight ranges greater than 20,000 g/mol.

### Example X

The synthesis of Example 1 is repeated except for the substitution of any polyoxyethylated polyol (POG) for polyethylene glycol (PEG).

### Example XI

The synthesis of Example 1 is repeated except for the substitution of polypropelene glycol (PPG) for polyethylene glycol (PEG).

### Example XII

The synthesis of Example 1 is repeated except for the substitution of oxidase type enzymes. pH range for oxidase enzymes is kept between 5 and 8.

### Example XIII

The synthesis of Example III is repeated except for the substitution of the polymer used in any of previous examples for PCL.

### Example XIV

The synthesis in Example IV is repeated except for the substitution of any of the polymers used in previous examples for PCL.

### Example XV

The synthesis in Example VI is repeated except for the substitution of any of the polymers used in previous examples for PCL.

### Example XVI

The synthesis in Example VI is repeated except for the substitution of any of the polymers used in previous examples for PCL.

### Example XVII

The synthesis in Example VII is repeated except for the substitution of any enzymes previously mentioned for protease.

### Example XVIII

The synthesis in Example VIII is repeated in any example provided as an extra step for added stability and uniformity of materials.

### Example XIX

The synthesis in Example XII is repeated except for the substitution of any polymer mentioned or silica/sand particles for PCL.
Electrospinning does not need to be done for any of these examples, but is an optional step depending on final usage of conjugate.
All polymers and enzymes here are interchangeable when paired correctly relative to their appropriate mw, such as described herein for PEG.

### Example XX

The synthesis in Example I is repeated except for the substitution of Tungsten or any other transition metal nanoparticles for silver nanoparticles.

### Example XXI

The synthesis in Example I is repeated except for the substitution of graphene for hBN as the 2d nanomaterial component.

### Example XXII

The synthesis in Example II is repeated except for the substitution of Tungsten or any other transition metal nanoparticles for silver nanoparticles.

### Example XXIII

The synthesis in Example II is repeated except for the substitution of Tungsten or any other transition metal nanoparticles for silver nanoparticles.

What is claimed is:

1. In a process for synthesis of an enzyme-polymer conjugate wherein each of said enzyme and polymer have an affinity for site specific binding of said enzyme to said polymer and thereby form an enzyme-polymer conjugate, an improved method comprising the steps of:
A. dissolving an amount of the enzyme in a first buffer solution to a concentration not exceeding the saturation of the said first buffer solution;
B. adding a polymer pair to said enzyme in said first buffer solution, in an equimolar amount, based upon said polymer pair affinity for site specific binding to said enzyme, and not exceeding the solubility of said polymer pair in said first buffer solution;
C. cross-linking of said enzyme and polymer pair in said first buffer solution by mildly agitating said first buffer solution and its contents under an inert atmosphere and at a depressed temperature sufficient to attain the maximum density of the first buffer solution, for an interval sufficient to attain equilibrium of site specific binding of said enzyme and said polymer pair, and thereby form a conjugate between said enzyme and polymer pair, without substantial steric hindrance or chemical interference of enzyme activity of said enzyme-polymer conjugate;
D. dissolving an additional amount of said polymer pair in a second buffer solution, in essentially the same equimolar amount as in Step B, and not to exceed the solubility of said additional polymer pair in said second buffer solution, wherein the volume of said second buffer solution is approximately at least twice the volume as said first buffer solution in Step A; and
E. mixing said combined solution of step D with mild agitation and in an inert atmosphere for an interval sufficient to effect an equilibrium cross-linking of said enzyme and polymer pair, thereby forming additional conjugate between said enzyme and polymer pair, without substantial steric hindrance or chemical interference of enzyme activity of said enzyme-polymer conjugate.

2. The improved process of claim 1, wherein said polymer pair has multiple binding sites for each binding site on said enzyme.

3. The improved process of claim 1, comprising an additional step of:
F. dissolving an additional amount of said polymer pair in a third buffer solution, in essentially the same equimolar amount as said polymer pair of Step B and mixing said third polymer solution with said combined solution of Step E for an interval sufficient to effect a cross-linking equilibrium of said enzyme and polymer pair, thereby forming additional enzyme-polymer conjugate between said enzyme and polymer pair.

4. The improved process of claim 1 wherein said enzyme is selected from the group consisting of exhibiting the functionality of proteinases, kinases, proteases, laccases, peroxidases, polymerases/transferases, oxidoreductases, and mixtures thereof.

5. The improved process of claim 1 wherein said polymer pair is a homo-polymer selected from the group consisting of aldoximes, ketoximes, muco-adhesion monomers, polyethylene glycol, bis-pyridinium oximes, N,N-dimethylacrylamide, N-isopropylacrylamide, (meth)acrylate, N,N-dimethylaminoethyl methacrylate, carboxyl acrylamide, 2-hydroxylethylmethacrylate, N-(2-hydroxypropyl)meth-acrylamide, quaternary ammonium monomers, sulfobetain methacrylate, oligo(ethylene glycol)methyl ether methacrylate, 2-PAM monomers, 4-PAM monomers, Clickable azide monomers, and mixtures thereof.

6. The improved process of claim 1 wherein said polymer pair is polycaprolactone (PCL)

7. The improved process of claim 1 wherein said polymer pair is polyethylene glycol. (PEG)

8. The improved process of claim 1 wherein said polymer pair is polyethylene glycol methacrylate (PEGMA)

9. The improved process of claim 1 wherein said polymer pair is polyacrylonitrile (PAN)

10. The improved process of claim 1 wherein said polymer pair is polyoxyethylated polyol (POG).

11. The improved process of claim 1 wherein said polymer pair is a co-polymer comprising at least one monomer selected from the group consisting of at least one monomer comprises aldoximes, ketoximes, muco-adhesion monomers, polyethylene glycol, bis-pyridinium oximes, N,N-dimethylacrylamide, N-isopropylacrylamide methacrylate, N,N-dimethylaminoethyl methacrylate, carboxyl acrylamide, 2-hydroxylethylmethacrylate, N-(2-hydroxypropyl) methacrylamide, quaternary ammonium monomers, sulfobetain methacrylate, oligo(ethylene glycol)methyl ether methacrylate, 2-PAM monomers, 4-PAM monomers, Clickable azide monomers, and combinations thereof.

12. A stable enzymatically active conjugate of the improved process of claim 2 wherein said enzyme-polymer conjugate has proteinases activity that is essentially free of steric hindrance and/or chemical interference of said proteinases activity of said conjugate, by site specific binding of said enzyme with said polymer pair.

13. A stable enzymatically active conjugate of the improved process of claim 2 wherein said enzyme-polymer conjugate has kinases activity that is essentially free of steric hindrance and/or chemical interference of said kinases activity of said conjugate, by site specific binding of said enzyme with said polymer pair

14. A stable enzymatically active conjugate of the improved process of claim 2 wherein said enzyme-polymer conjugate has protease activity that is essentially free of steric hindrance and/or chemical interference of said protease activity of said conjugate, by site specific binding of said enzyme with said polymer pair

15. A stable enzymatically active conjugate of the improved process of claim 2 wherein said enzyme-polymer conjugate has peroxidases activity that is essentially free of steric hindrance and/or chemical interference of said peroxidases activity of said conjugate, by site specific binding of said enzyme with said polymer pair

16. A stable enzymatically active conjugate of the improved process of claim 2 wherein said enzyme-polymer conjugate has laccases activity that is essentially free of steric hindrance and/or chemical interference of said laccases activity of said conjugate, by site specific binding of said enzyme with said polymer pair,

17. A stable enzymatically active conjugate of the improved process of claim 2 wherein said enzyme-polymer conjugate has polymerases/transferases activity that is essentially free of steric hindrance and/or chemical interference of said polymerases/transferases activity of said conjugate, by site specific binding of said enzyme with said polymer pair

18. A stable enzymatically active conjugate of the improved process of claim 2 wherein said enzyme-polymer conjugate has oxidoreductase activity that is essentially free of steric hindrance and/or chemical interference of said oxidoreductase activity of said conjugate, by site specific binding of said enzyme with said polymer pair.

19. An improved process for the synthesis of an enzyme-polymer conjugate wherein each of said enzyme and polymer have an affinity for site specific binding of said enzyme to said polymer, and thereby form an enzyme-polymer conjugate, comprising the steps of:
A. dissolving an amount of enzyme in a first buffer solution, not to exceed the solubility of said enzyme in said first buffer solution;
B. adding a polymer pair to said enzyme in said first buffer solution, in an equimolar amount, based upon said polymer pair affinity for site specific binding to said enzyme, and not to exceed the solubility of said polymer pair in said first buffer solution, said polymer pair being further characterized as a coordination compound wherein, said coordination compound comprise repeating units of following formula:
(-[-Enzyme-Coordination Polymer-tmMOF-Coordination Polymer]ₙ-) wherein tm is transition metal (e.g. silver) within a metal organic framework of the coordination polymer,
C. cross-linking of said enzyme and polymer pair, in said first buffer, by mildly agitating said first buffer and its contents under an inert atmosphere, and at a depressed temperature sufficient to attain the maximum density of the first buffer, for an interval sufficient to attain equilibrium of site specific binding of said enzyme and said polymer to form a conjugate between said enzyme and polymer pair, without substantial steric hindrance or chemical interference of enzyme activity of said enzyme-polymer conjugate;
D. dissolving an additional amount of said polymer pair in a second buffer solution, in essentially the same equimolar amount as in Step B, and not to exceed the solubility of said additional polymer pair in said second buffer solution, wherein the volume of said second buffer is approximately at least twice the volume as said first buffer solution in Step A; and
E. mixing said combined solution of step D, with mild agitation and in an inert atmosphere, for an interval sufficient to effect a equilibrium cross-linking of said enzyme and polymer pair, thereby forming additional conjugate between said enzyme and polymer pair, without substantial steric hindrance or chemical interference of enzyme activity of said enzyme-polymer conjugate.

20. The improved process of claim 19 wherein said polymer pair has multiple binding sites for each binding site on said enzyme.

21. The improved process of claim 19 comprising an additional Step F wherein an additional amount of said polymer pair is dissolved in a third buffer solution, in essentially the same equimolar amount as said polymer pair of Step B, and mixing said third polymer solution, with said combined solution of Step E, for an interval sufficient to effect a cross-linking equilibrium of said enzyme and polymer pair, thereby forming additional enzyme-polymer conjugate between said enzyme and polymer pair.

22. The improved process of claim 19 wherein said enzyme is selected from the group consisting essentially of exhibiting proteinases, kinases, proteases, laccases, peroxidases, polymerases/transferases, oxidoreductase functionality and mixtures thereof.

23. The improved process of claim 19 wherein said coordination polymer pair is a homo-polymer selected from the group consisting of aldoximes, ketoximes, muco-adhesion monomers, polyethylene glycol, bis-pyridinium oximes, N,N-dimethylacrylamide, N-isopropylacrylamide, (meth)acrylate, N,N-dimethylaminoethyl methacrylate, carboxyl acrylamide, 2-hydroxylethylmethacrylate, N-(2-hydroxypropyl)meth-acrylamide, quaternary ammonium monomers, sulfobetain methacrylate, oligo(ethylene glycol)methyl ether methacrylate, 2-PAM monomers, 4-PAM monomers, Clickable azide monomers, and mixtures thereof.

24. The improved process of claim 19 wherein said coordination polymer pair is polycaprolactone (PCL)

25. The improved process of claim 19 wherein said coordination polymer pair is polyethylene glycol.(PEG)

26. The improved process of claim 19 wherein said coordination polymer pair is polyethylene glycol methacrylate (PEGMA)

27. The improved process of claim 19 wherein said coordination polymer pair is polyacrylonitrile (PAN)

28. The improved process of claim 19 wherein said coordination polymer pair is polyoxyethylated polyol (POG).

29. The improved process of claim 19 wherein said coordination polymer pair is a co-polymer comprising at least one monomer selected from the group consisting of at least one monomer comprises aldoximes, ketoximes, muco-adhesion monomers, polyethylene glycol, bis-pyridinium oximes, N,N-dimethylacrylamide, N-isopropylacrylamide methacrylate, N,N-dimethylaminoethyl methacrylate, carboxyl acrylamide, 2-hydroxylethylmethacrylate, N-(2-hydroxypropyl) methacrylamide, quaternary ammonium monomers, sulfobetain methacrylate, oligo(ethylene glycol)methyl ether methacrylate, 2-PAM monomers, 4-PAM monomers, Clickable azide monomers, and combinations thereof.
